(19) United States
(12) Patent Application Publication
(10) Pub. No.: US 2026/0123842 A1
(43) Pub. Date: May 7, 2026

(54) SYSTEM AND METHOD FOR NON-INVASIVE CARDIOVASCULAR HEALTH ASSESSMENT

(71) Applicant: Hong Kong Centre for Cerebro-Cardiovascular Health Engineering Limited, Pak Shek Kok (HK)

(72) Inventors: Zainab Jamil, Pak Shek Kok (HK); Ho Man Chan, Pak Shek Kok (HK); Liangyi Lyu, Pak Shek Kok (HK); Leong Ting Lui, Pak Shek Kok (HK); Zheng Gong, Pak Shek Kok (HK)

(21) Appl. No.: 19/372,569

(22) Filed: Oct. 29, 2025

Related U.S. Application Data
(60) Provisional application No. 63/717,343, filed on Nov. 7, 2024.

(51) Int. Cl. A61B 5/0205; A61B 5/00; A61B 5/346

(52) U.S. Cl. CPC A61B 5/0205; A61B 5/346; A61B 5/7235; A61B 5/742; A61B 2560/0462; A61B 2562/063

(57) ABSTRACT

A computer-implemented method for non-invasive Cardiovascular health assessment including, receiving, at a processor, physiological signals from a subject, wherein the physiological signals comprising at least electrocardiogram (ECG) signals and photoplethysmogram (PPG) signals, processing, by an artificial intelligence (AI) model executed by the processor, the ECG signals and PPG signals, and; estimating, by the AI model, one or more demographic and anthropometric parameters based on processing the ECG and PPG signals.

### TECHNICAL FIELD

The present invention relates to a system and method for non-invasive cardiovascular health assessment. The present invention may also relate to a system and method for non-invasive cardiovascular health assessment based on ECG and PPG signals.

### BACKGROUND

Cardiovascular health remains a central challenge in contemporary medicine, as it directly influences both life expectancy and quality of life. Accurate estimation of demographic and anthropometric features such as age, gender, height, weight, and body mass index (BMI) is not only critical for effective clinical monitoring but also for early detection of cardiovascular disease (CVD) risk, individualized prevention strategies, and tailored treatment plans. Despite their importance, current methods for acquiring these parameters are limited. Manual anthropometric measurements are subject to human error, inter-operator variability, and device-specific inconsistencies, making them unreliable in large-scale or remote healthcare settings. Invasive or laboratory-based measurements, while precise, are impractical for routine monitoring due to their associated discomfort, risks, and time requirements.

Even where electrocardiogram (ECG) and photoplethysmogram (PPG) are employed in practice, their use is largely confined to blood pressure (BP) estimation, arrhythmia detection, or pulse analysis, with no established frameworks for demographic or anthropometric prediction. Furthermore, many existing AI systems in healthcare suffer from bias and fairness concerns, leading to uneven prediction quality across gender, age, or population subgroups. There is a need for an improved system that addresses the shortcomings of conventional methods of determining demographic and anthropometric parameters.

### SUMMARY

The present invention seeks to provide a system and method for non-invasive cardiovascular health assessment, which will overcome or substantially ameliorate at least some of the deficiencies of the prior art, or to at least provide an alternative.

In accordance with a first aspect, there is provided a computer-implemented method for non-invasive cardiovascular health assessment comprising:
receiving, at a processor, physiological signals from a subject,
wherein the physiological signals comprising at least electrocardiogram (ECG) signals and a photoplethysmogram (PPG) signals,
processing, by an artificial intelligence (AI) model executed by the processor, the ECG signals and PPG signals, and;
estimating, by the AI model, one or more demographic and anthropometric parameters based on processing the ECG and PPG signals.

In one example, the step of processing comprising:
pre-processing the received ECG signals and PPG signals,
performing feature extraction of the pre-processed ECG signals and PPG signals,
selecting one or more optimal features, and;
wherein the demographic and anthropometric parameters are estimated based on the one or more optimal features.

In one example, the step of processing comprising the step of performing temporal processing of the pre-processed ECG signals and PPG signals.

In one example, temporal processing comprising capturing temporal dependencies and spatial relationships between the one or more optimal features.

In one example, the demographic parameters comprising age, gender and the one or more anthropometric features comprising weight, height and BMI.

In one example, the method further comprising
calculating, by the AI model, a cardiovascular age for the subject based on the estimated demographic and anthropometric parameters, and;
outputting the cardiovascular age and/or comparing the calculated cardiovascular age to the subject's chronological age to identify a risk of vascular aging.

In one example, the method comprising tracking changes in estimated BMI or weight over time to detect abnormal trends and presenting estimated BMI or weight changes on a user interface.

In one example, the method further comprising:
generating a visual indicator to illustrate the estimate of the demographic and anthropomorphic parameters,
presenting the visual indicator on a user interface, and;
wherein the visual indicator comprising one or more of: a saliency map or heat map or attention heat map.

In one example, the AI model comprising a recurrent neural network (RNN model), wherein the RNN model is trained to perform the steps of processing the received ECG and PPG signals and output an estimate of one or more demographic and anthropometric parameters.

In one example, the method may optionally apply ensemble methods to combine predictions from diverse model architectures. This can improve robustness and reliability.

In accordance with a further aspect, there is provided a system for non-invasive cardiovascular health monitoring comprising:
a computing apparatus comprising a processor and a memory unit, the processor and memory unit being operatively coupled to each other,
a user interface operatively coupled to or integrated into the computing apparatus,
the memory unit adapted to store an artificial intelligence (AI) model, wherein the AI model is executable by the processor, the memory unit further comprising instructions which, when executed by the processor cause the processor to:
   receive physiological signals from a subject,
   wherein the physiological signals comprising at least electrocardiogram (ECG) signals and a photoplethysmogram (PPG) signals,
   process, by the AI model, the ECG signals and PPG signals, and;
   estimate, by the AI model, one or more demographic and anthropometric parameters based on processing the ECG and PPG signals, and;
display the estimated demographic and anthropometric parameters on the user interface.

In one example, the user interface may be a screen e.g., an LED or LCD screen. Optionally, the screen may be touchscreen.

In one example, the processor is programmed to:
pre-process the received ECG signals and PPG signals,
perform feature extraction of the pre-processed ECG signals and PPG signals,
select one or more optimal features, and;
wherein the demographic and anthropometric parameters are estimated based on the one or more optimal features.

In one example, the processor is programmed to perform temporal processing of the pre-processed ECG signals and PPG signals.

In one example, during temporal processing the processor is programmed to capture temporal dependencies and spatial relationships between the one or more optimal features.

In one example, the demographic parameters comprise age, gender and the one or more anthropometric features comprising weight, height and BMI.

In one example, the processor is programmed to:
calculate, by applying the AI model, a cardiovascular age for the subject based on the estimated demographic and anthropometric parameters; and
output the cardiovascular age and/or comparing the calculated cardiovascular age to the subject's chronological age to identify a risk of vascular aging.

In one example, the processor is programmed to track changes in estimated BMI or weight over time to detect abnormal trends and present estimated BMI or weight changes on the user interface.

In one example, the processor is further programmed to:
generate a visual indicator (i.e., visual indicia) to illustrate the estimate of the demographic and anthropomorphic parameters,
present the visual indicator (i.e., visual indicia) on the user interface, and;
wherein the visual indicator comprising one or more of: a saliency map or heat map or attention heat map.

In one example, the visual indicator may be displayed on the user interface.

In one example, the AI model comprising a recurrent neural network (RNN model), wherein the RNN model is trained to perform the steps of processing the received ECG and PPG signals and output an estimate of one or more demographic and anthropometric parameters.

In one example, the AI model is pretrained using a self-supervised learning framework on a dataset of unlabeled ECG signals, PPG signals, or both and, wherein the self-supervised learning framework is a masked self-supervised learning framework wherein segments of the unlabeled signals are masked and the AI model is trained to reconstruct the masked segments.

In one example, the AI model may further comprise:
an encoder that is trained to extract features from the ECG and/or the PPG signals,
one or more task specific classification heads applied for regression to estimate an anthropometric parameter and/or classification to estimate a demographic parameter,
wherein the encoder is coupled to the one or more task specific classification heads such that the output of the encoder is the input to each of the one or more classification heads, and the encoder is upstream of each of the one or more classification heads.

In one example, the encoder may be pretrained. In one example, the encoder may be a PatchTST encoder.

In one example, the AI model may be pretrained by employing a self-supervised learning method. The pretraining provides generalized feature representations that can be transferred to multiple downstream tasks without requiring full retraining.

In one example, the AI model may optionally comprise an ensemble architecture that combines multiple predictions from diverse model architectures, and; wherein the multiple model architectures may define the AI model. This can improve robustness and reliability.

In accordance with a further aspect, there is provided a wearable device for non-invasive cardiovascular health monitoring, comprising:
a sensor array configured to continuously acquire ECG and PPG signals from a user;
a computing apparatus, the computing apparatus comprising a processor and a memory unit, the processor and memory unit being operatively coupled to each other,
the computing apparatus being operatively coupled to the sensor array,
the memory unit adapted to store an artificial intelligence (AI) model, wherein the AI model is executable by the processor, the memory unit further comprising instructions which, when executed by the processor cause the processor to:
   receive physiological signals from a subject,
   wherein the physiological signals comprising at least electrocardiogram (ECG) signals and a photoplethysmogram (PPG) signals,
   process, by the AI model, the ECG signals and PPG signals, and;
   estimate, by the AI model, one or more demographic and anthropometric parameters based on processing the ECG and PPG signals.

In one example, the wearable device may comprise a user interface or the wearable device may be arranged in communication with a user interface. The one or more estimated demographic parameters and one or more estimated anthropometric parameters may be presented on the user interface.

In accordance with a further aspect, there is provided an artificial intelligence (AI) model for non-invasive cardiovascular health assessment, for use in the method of as describe in the first aspect, comprising:
a machine learning model or deep learning model for feature extraction and selecting optimal features,
a recurrent neural network or a transformer for temporal processing of the optimal features,
wherein the AI model is trained to process ECG signals and PPG signals, and;
wherein the AI model is trained to estimate one or more demographic and anthropometric parameters based on processing the ECG and PPG signals.

In one example, the AI model comprising an ensemble of multiple deep learning models with diverse architectures, and wherein the estimation of demographic and anthropometric parameters are generated by combining outputs from the multiple deep learning models.

In one example, the AI model is trained using self-supervised learning framework on a dataset of unlabeled ECG signals and PPG signals, wherein the self-supervised learning framework is a masked self-supervised learning framework wherein segments of the unlabeled signals are masked and the AI model is trained to reconstruct the masked segments.

In one example, the AI model comprising:
an encoder that is trained to extract features from the ECG and/or the PPG signals,
one or more task specific classification heads applied for regression to estimate an anthropometric parameter and/or classification to estimate a demographic parameter,
wherein the encoder is coupled to the one or more task specific classification heads such that the output of the encoder is the input to each of the one or more classification heads, and the encoder is upstream of each of the one or more classification heads.

In one example, the encoder may be pretrained. In one example, the encoder may be a PatchTST encoder.

In accordance with a further aspect, there is provided a data processing apparatus for use in non-invasive cardiovascular health monitoring comprising means for carrying out the method as described herein.

In accordance with a further aspect, there is provided a computer program comprising instructions which, when the program is executed by a computer, cause the computer to carry out the method according to the first aspect (i.e., described in the first aspect).

In accordance with a further aspect, there is provided a computer-readable medium comprising instructions which, when executed by a computer, cause the computer to carry out the method according to the first aspect.

In accordance with a further aspect, there is provided a system for non-invasive cardiovascular health assessment, the system comprising:
one or more processors; and
a memory storing instructions that, when executed by the one or more processors, cause the system to:
   receive physiological signals from a subject, wherein the physiological signals comprise at least an electrocardiogram (ECG) signal and a photoplethysmogram (PPG) signal;
   pre-process the received ECG and PPG signals to reduce noise and normalize the signals;
   input the pre-processed signals into an artificial intelligence (AI) model, the AI model comprising a deep learning architecture; and
   generate, using the AI model, an estimation of a plurality of demographic and anthropometric parameters of the subject, the parameters including age, gender, height, weight, and body mass index (BMI), based on a detailed analysis of temporal and spatial patterns within the pre-processed signals.

In one example, the deep learning architecture is a recurrent neural network (RNN).

In one example, the deep learning architecture is a transformer-based architecture.

In one example, the AI model is pretrained using a self-supervised learning framework on a dataset of unlabeled ECG signals, PPG signals, or both.

In one example, the self-supervised learning framework is a masked self-supervised learning framework wherein segments of the unlabeled signals are masked and the AI model is trained to reconstruct the masked segments.

In one example, the AI model comprises an ensemble of multiple deep learning models with diverse architectures, and wherein the estimations are generated by combining outputs from the multiple deep learning models.

In one example, the instructions further cause the system to integrate additional sensor data with the ECG and PPG signals, the additional sensor data selected from the group consisting of a respiration signal, an oxygen saturation (SpO2) signal, and an accelerometer signal.

In one example, the instructions further cause the system to implement an explainable AI (XAI) module to provide an interpretable basis for the generated estimations, wherein the XAI module generates at least one of a saliency map or an attention heatmap.

In one example, the system is integrated into a wearable device, and wherein the instructions further cause the system to perform real-time, on-device inference to generate the estimations.

In one example, the AI model is optimized for deployment on the wearable device using one or more compression techniques selected from the group consisting of model quantization, pruning, and bias-only fine-tuning.

In one example, the instructions further cause the system to perform longitudinal tracking by repeatedly generating the estimations over time to monitor changes in at least one of the subject's estimated weight, BMI, or a calculated cardiovascular age.

In accordance with a further aspect, there is provided a method for non-invasive cardiovascular health assessment, the method comprising:
receiving, at one or more processors, physiological signals from a subject, the physiological signals comprising at least an electrocardiogram (ECG) signal and a photoplethysmogram (PPG) signal;
pre-processing, by the one or more processors, the received ECG and PPG signals to ensure signal quality and consistency;
analyzing, by an artificial intelligence (AI) model executed on the one or more processors, temporal and spatial features extracted from the pre-processed signals; and;
estimating, by the AI model, a plurality of the subject's demographic and anthropometric parameters, wherein the parameters comprise age, gender, height, weight, and body mass index (BMI).

In one example, the method further comprising pre-training the AI model on a dataset of unlabeled physiological signals using a self-supervised learning framework prior to the analyzing step.

In one example, the AI model comprises a transformer-based architecture configured to capture time-dependent patterns in the pre-processed signals.

In one example, the method further comprising training and updating the AI model using a federated learning framework, thereby preserving data privacy across multiple data sources.

In one example, the method further comprising applying an ethical bias-checking mechanism to the AI model to promote fairness and mitigate prediction disparities across different population subgroups.

In one example, the method further comprising:
calculating a cardiovascular age for the subject based on the estimated demographic and anthropometric parameters, and;
comparing the calculated cardiovascular age to the subject's chronological age to identify a risk of vascular aging.

In one example the method may be performed by a telemedicine platform to facilitate remote patient monitoring.

In one example, the method comprising generating a personalized healthcare recommendation for the subject based on the estimated parameters.

In accordance with a further aspect, there is provided a system for non-invasive cardiovascular health assessment including:
methods for collecting pre-processed ECG and PPG signals from subjects.
an AI-based combining ML/DL approaches and recurrent neural networks (RNNs) or transformer architecture for feature extraction and temporal learning.
stated deep learning model predicting age, gender, height, weight, and BMI of subjects based on the pre-processed signals, wherein the deep learning model's architecture enables detailed analysis of temporal and spatial signal patterns.

In one example, the system assists with maintaining the quality, alignment, and consistency of the ECG and PPG signals acquired from the available online CVD datasets. The ML/DL model in the stated hybrid approach will extract features from the ECG and PPG signals.

In one example, wherein the DL/ML model incorporates a recurrent neural network (RNN) component or transformer to capture temporal dependencies and spatial relationships within the signals. This combination enables the system to perform a thorough estimate of cardiovascular parameters.

In one example, the system further comprises an ensemble learning mechanism, wherein multiple deep AI-based with diverse architectures are combined to improve the accuracy and robustness of age, gender, height, weight, and BMI estimations.

In one example, the system may be enhanced with an ethical bias-checking mechanism to promote fairness and ensure clinically sound parameter estimations. Additionally, the system addresses potential disparities in predictions.

In one example, the system may be integrated into existing healthcare systems, telemedicine platforms, and wearable health devices for real-time cardiovascular health assessment and remote patient monitoring, facilitating proactive healthcare management.

In one example, the system may be validated through clinical trials, acquiring regulatory approvals as a non-invasive cardiovascular health assessment technology, thereby advancing healthcare diagnostics and enhancing patient outcomes.

In accordance with a further aspect, there is provided a system for non-invasive cardiovascular health assessment including:
methods for collecting pre-processed ECG and PPG signals from subjects.
an AI-based combining ML/DL approaches and recurrent neural networks (RNNs) or transformer architecture for feature extraction and temporal learning.
a DL (deep learning) model predicting age, gender, height, weight, and BMI of subjects based on the pre-processed signals, wherein the DL model's architecture enables detailed analysis of temporal and spatial signal patterns.

In one example, the quality, alignment, and consistency of the ECG and PPG signals are ensured from the available online cardiovascular disease (CVD) datasets, and wherein the hybrid ML/DL model extracts features from the signals.

In one example, the DL/ML model incorporates a recurrent neural network (RNN) component or transformer to capture temporal dependencies and spatial relationships within the signals. This combination enables the system to perform a thorough estimate of cardiovascular parameters.

In one example, the system comprises an ensemble learning mechanism, wherein multiple deep AI-based with diverse architectures are combined to improve the accuracy and robustness of age, gender, height, weight, and BMI estimations.

In one example, the system may be enhanced with an ethical bias-checking mechanism to promote fairness and ensure clinically sound parameter estimations. Additionally, the system addresses potential disparities in predictions.

In one example, the system may be integrated into existing healthcare systems, telemedicine platforms, and wearable health devices for real-time cardiovascular health assessment and remote patient monitoring, facilitating proactive healthcare management.

In one example the AI model may be validated through clinical trials, acquiring regulatory approvals as a non-invasive cardiovascular health assessment technology, thereby advancing healthcare diagnostics and enhancing patient outcomes.

In one example the AI model is pretrained using a self-supervised learning framework on unlabeled ECG and/or PPG signals, followed by evaluation for demographic parameter estimation.

In one example, the AI model incorporates explainable AI modules, including saliency mapping, attention visualization, or equivalent interpretability methods, to provide clinicians with interpretable reasoning for each estimation.

In one example, comprising a longitudinal tracking mechanism to monitor changes in estimated weight, BMI, or cardiovascular age over time, thereby enabling early detection of abnormal health trends.

In one example, the model is optimized for deployment on wearable devices through compression techniques including model quantization, pruning, or bias-only fine-tuning, enabling real-time on-device inference.

In one example, model training and updating is performed using a federated learning framework, thereby preserving data privacy while aggregating knowledge across multiple devices or institutions.

In one example additional sensor modalities such as respiration, SpO₂, or accelerometer signals are integrated alongside ECG and PPG to improve robustness of demographic estimation in ambulatory or wearable settings.

The term “comprising” (and its grammatical variations) as used herein are used in the inclusive sense of “having” or “including” and not in the sense of “consisting only of”.

It is to be understood that, if any prior art information is referred to herein, such reference does not constitute an admission that the information forms a part of the common general knowledge in the art, in Australia or any other country

### BRIEF DESCRIPTION OF THE DRAWINGS

Embodiments of the present invention will now be described, by way of example, with reference to the accompanying drawings in which:

FIG. 1 illustrates a schematic diagram of an example embodiment of a system for non-invasive cardiovascular health assessment.

FIG. 2 illustrates a flow chart of an example embodiment of a method for non-invasive cardiovascular health assessment.

FIG. 3 illustrates a flow chart of another example embodiment of a method for non-invasive cardiovascular health assessment.

FIG. 4 illustrates a schematic diagram of an example wearable device for use in non-invasive cardiovascular health assessment or cardiovascular monitoring

FIG. 5 illustrates an example architecture of the AI model used in non-invasive cardiovascular health assessment.

FIG. 6 illustrates an example of architecture of the PatchTST encoder in the AI model.

FIG. 7 illustrates a training and validation process for the AI model used for non-invasive cardiovascular health assessment.

FIG. 8 illustrates a further alternative embodiment of a method of non-invasive cardiovascular health assessment

### DETAILED DESCRIPTION

Cardiovascular health is a cornerstone of overall well-being, and the accurate estimation of demographic and anthropometric parameters such as age, gender, height, weight, and body mass index (BMI) plays a critical role in effective healthcare management. Conventional methods for obtaining these parameters are often invasive, time-consuming, or prone to error, highlighting the need for non-intrusive alternatives.

The rapid growth of wearable technologies, such as smartwatches, chest straps, and fingertip PPG sensors, has made continuous and non-invasive cardiovascular monitoring both feasible and scalable. The clinical implications of accurate demographic estimation from ECG and PPG are significant. Age is directly linked with arterial stiffness and the onset of CVD; abnormal weight, BMI, or height patterns can signal elevated risk of hypertension, obesity, and metabolic disorders; and gender differences are known to influence cardiovascular outcomes. The increasing availability of large public ECG and PPG datasets enables robust data-driven modelling.

The present invention relates to a system and method for non-invasive cardiovascular health monitoring. The invention may also relate to a wearable device that includes an integrated computing apparatus that enables non-invasive cardiovascular health monitoring. The present invention relates to a system, method and/or wearable device that incorporates machine learning or other AI approaches for cardiovascular health assessment by estimating demographic and/or anthropometric features from non-invasively measured signals.

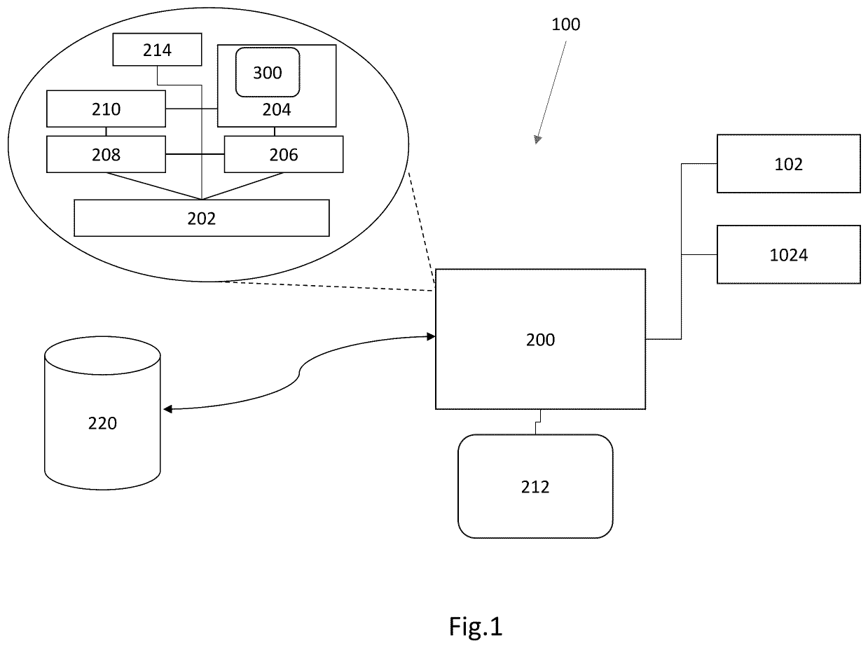

FIG. 1 illustrates an example of a system 100 for non-invasive cardiovascular health monitoring. Referring to figure the system 100 for non-invasive cardiovascular health monitoring comprising: a computing apparatus 200 comprising a processor 202 and a memory unit 204, 206, the processor and memory unit being operatively coupled to each other, and a user interface 212 operatively coupled to or integrated into the computing apparatus 200. The memory unit 204, 206 adapted to store an artificial intelligence (AI) model 300, wherein the AI model is executable by the processor 202. The memory unit 204, 206 further comprising instructions which, when executed by the processor 202 cause the processor to: receive physiological signals from a subject, wherein the physiological signals comprising at least electrocardiogram (ECG) signals and a photoplethysmogram (PPG) signals, process, by the AI model 300, the ECG signals and PPG signals, and; estimate, by the AI model, one or more demographic and anthropometric parameters based on processing the ECG and PPG signals, and; display the estimated demographic and anthropometric parameters on the user interface 212.

The system 100 integrates electrocardiogram (ECG) and photoplethysmogram (PPG) signals with advanced artificial intelligence (AI) techniques to predict key demographic features with high accuracy. The approach employs signal preprocessing for noise reduction and normalization, followed by machine learning and deep learning architectures applied in the AI model. The machine learning and deep learning architectures can include recurrent neural networks and transformer models, for automated feature extraction and temporal learning.

Age is directly linked with arterial stiffness and the onset of cardiovascular disease. The demographic and anthropometric parameters estimated by the AI model 300 provide an indication of cardiovascular health of the subject. Abnormal weight, BMI, or height patterns can signal elevated risk of cardiovascular disease or hypertension or other conditions and are generally indicative of poor cardiovascular health. The system enables accurate, real-time, and non-invasive prediction of these features to allow non-invasive assessment of cardiovascular health of a subject.

The system 100 may comprise one or more sensors. As shown in the example configuration of FIG. 1, the system 100 may comprise an ECG sensor 102 and a PPG sensor 104. The sensors 102, 104 are arranged in electrical communication with the computing apparatus 200. The computing apparatus 200 can receive ECG signals and PPG signals from sensors 102, 104 respectively. The ECG sensor may comprise a chest band or a plurality of electrodes. The PPG sensor may be fingertip PPG sensor or a wrist mounted sensor such as for example a pulse oximeter.

The computing apparatus 200 may be implemented by a computer having an appropriate user interface. The computing apparatus 200 may be implemented by any computing architecture, including portable computers, tablet computers, stand-alone Personal Computers (PCs), smart devices, Internet of Things (IOT) devices, edge computing devices, client/server architecture, “dumb” terminal/mainframe architecture, cloud-computing based architecture, or any other appropriate architecture. The computing apparatus 200 may be appropriately programmed to implement a method for non-invasive cardiovascular health monitoring as described herein.

The computing apparatus 200 may be implemented as a general-purpose processor, a digital signal processor (DSP), an application specific integrated circuit (ASIC), a field programmable gate array (FPGA) or other programmable logic component, discrete gate or transistor logic, discrete hardware components, or any combination thereof designed to perform the functions described herein. The computing apparatus 200 may be programmed to implement a method for non-invasive cardiovascular health monitoring as described herein.

A general-purpose processor may be a microprocessor, but in the alternative, the processor may be any conventional processor, controller, microcontroller, circuit, and/or state machine. A processor may also be implemented as a combination of computing components, e.g., a combination of a DSP and a microprocessor, a number of microprocessors, one or more microprocessors in conjunction with a DSP core, or any other such configuration. In one example, the computing apparatus 200 may be implemented as a microprocessor.

The computing apparatus 200 may be programmed to estimate one or more demographic parameters and/or anthropometric parameters. In one example, the demographic parameters comprise age, gender and the one or more anthropometric features comprising weight, height and BMI.

As shown in FIG. 1 there is a shown a schematic diagram a system 100 for non-invasive cardiovascular health assessment or health monitoring. The system includes or may be implemented by the computing apparatus 200. FIG. 1 further illustrates a schematic diagram of the components of the computing apparatus 200.

In the illustrated example, the computing apparatus 200 comprises suitable components necessary to receive, store and execute appropriate computer instructions. The components may include a processor 202 (i.e., a processing unit 202). The processor may include one or more of a Central Processing Unit (CPU), Math Co-Processing Unit (Math Processor), Graphic Processing Unit (GPUs) or Tensor processing unit (TPUs) for tensor or multi-dimensional array calculations or manipulation operations. The computing apparatus 200 further comprises a read-only memory (ROM) 204, random access memory (RAM) 206, and input/output devices such as disk drives 208, input devices 210 such as an Ethernet port, a USB port, etc.

The computing apparatus 200 may also comprise a user interface 212. The user interface 212 may be a display 212. The display 112 may be a liquid crystal display, a light emitting display or any other suitable display. The user interface 212 may be a screen e.g., an LED or LCD screen. Optionally, the screen may be touchscreen. The computing apparatus 200 may also comprise a communications links 114. The user interface 212 may be integrated with the computing apparatus or may be remote from the computing apparatus. In the remote arrangement, at least the processor 202 may be operatively coupled to the user interface 212 e.g., by a wired or wireless connection for communication of data.

The computing apparatus 200 may include instructions that may be included in ROM 204, RAM 206 or disk drives 208 and may be executed by the processor 202. There may be provided a plurality of communication links 214 which may variously connect to one or more computing devices such as a server, personal computers, terminals, wireless or handheld computing devices, Internet of Things (IoT) devices, smart devices, edge computing devices. At least one of a plurality of communications link 214 may be connected to an external computing network through a telephone line or other type of communications link.

The computing apparatus 200 may include storage devices such as a disk drive 208 which may encompass solid state drives, hard disk drives, optical drives, magnetic tape drives or remote or cloud-based storage devices. The computing apparatus 200 may use a single disk drive or multiple disk drives, or a remote storage service. The computing apparatus may also have a suitable operating system which resides a memory unit of the computing apparatus 200.

The computing apparatus 200 may further comprise one or more databases adapted to store one or more pieces of data. For example, the apparatus 200 may include a database 220 of training data e.g., ECG signals and PPG signals with corresponding demographic and anthropometric labels such as age, gender, height, weight and BMI. The datasets may be publicly available cardiovascular health assessment datasets that contain ECG and PPG signals. These datasets can provide the ground truth for the AI model during training to learn the correlations between biophysical signals and individual characteristics. The training dataset may be regularly updated with additional data to improve model performance.

The computing apparatus 200 may also provide the necessary computational capabilities to operate or to interface with an AI model 300. The AI model may be a machine learning network, such as a neural networks, to provide various functions and outputs. The AI model 300 may be implemented locally, or it may also be accessible or partially accessible via a server or cloud-based service. The AI model 300 may also be untrained, partially trained or fully trained, and/or may also be retrained, adapted or updated over time. The training may be updated using new training datasets.

The computing apparatus 200 may comprise one or more GPUs being operatively coupled to the CPU (i.e., processor). The computing apparatus 200 may comprise additional hardware elements operatively coupled to the CPU and/or the GPU to provide the computing apparatus components needed to implement an AI model such as for example a machine learning network or machine learning model. The AI model 300 may be stored in a memory unit e.g., ROM. The specifics of the AI model 300 will be described later.

The processor 202 is programmed to pre-process the received ECG signals and PPG signals, perform feature extraction of the pre-processed ECG signals and PPG signals, and select one or more optimal features. The processor 202 is further programmed to estimate the demographic and anthropometric parameters based on the one or more optimal features. The processor 202 and the computing apparatus is configured to estimate the demographic and anthropometric parameters based on the optimal features.

In one example the demographic parameters comprising age, gender and the one or more anthropometric features comprising weight, height and BMI. The processor 202 may execute the AI model 300 to estimate the demographic parameters and anthropometric parameters. The AI model 300 may be trained to estimate other demographic parameters and anthropometric parameters.

The processor 202 may also be programmed to perform temporal processing of the pre-processed ECG signals and PPG signals. The AI model 300 may be trained to perform temporal processing of the ECG and PPG signals. In one example, during temporal processing the processor is programmed to capture temporal dependencies and spatial relationships between the one or more optimal features.

The processor 202 may also be programmed to calculate, by applying the AI model, a cardiovascular age for the subject based on the estimated demographic and anthropometric parameters. The processor 202 may also be programmed to output the cardiovascular age and/or comparing the calculated cardiovascular age to the subject's chronological age to identify a risk of vascular aging.

In one example, the processor 200 may be programmed to track changes in estimated BMI or weight over time to detect abnormal trends and present estimated BMI or weight changes on the user interface. Additionally, the processor may be further programmed to generate a visual indicator (i.e., visual indicia) to illustrate the estimate of the demographic and anthropomorphic parameters. The processor 202 may also generate other visual indicators to display changes in demographic and anthropometric parameters.

The processor 202 may also be programmed to provide any one or more of: longitudinal tracking of health parameters to monitor aging progression or BMI fluctuations, cardiovascular age vs. chronological age comparison, enabling early identification of vascular aging and disease risk, or early detection of comorbidities, including hypertension, obesity, and predisposition to CVDs, based on demographic trends. The AI model 300 may be trained to output one or more of the described functions.

The processor 202 may also be programmed to display the functions as visual indicators. The processor 202 may be programmed to present the visual indicator (i.e., visual indicia) on the user interface. The visual indicator comprising one or more of: a saliency map or heat map or attention heat map. The visual indicator or indicators may be displayed on the user interface.

The system 100 may be integrated into real-world healthcare environments, including for example wearables or mobile devices, telemedicine platforms or cloud based or federated learning frameworks. Wearables and mobile devices may include for example as smartwatches, chest straps, or fingertip PPG devices for continuous monitoring. Integrating the system as part of a telemedicine platform allows for remote patient assessment and proactive care delivery. Integrating the system 100 into cloud-based or federated learning frameworks that preserve privacy, ensure HIPAA compliance, can enable population-level improvements without compromising patient data.

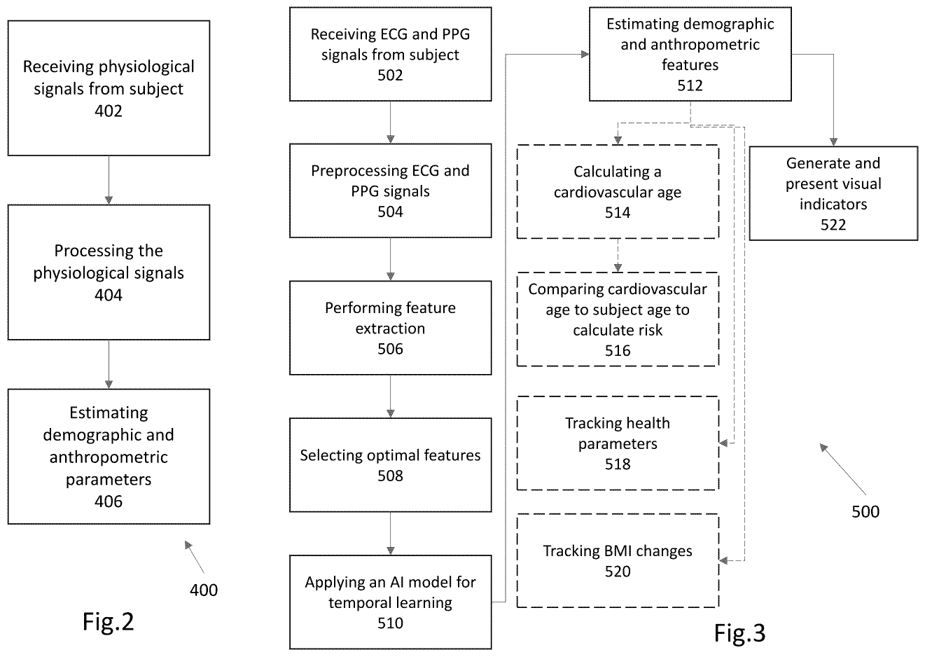

FIG. 2 illustrates an example of a computer-implemented method 400 for non-invasive cardiovascular health assessment. The method 400 may commence at step 402. Step 402 comprises receiving, at a processor, physiological signals from a subject 10. The physiological signals comprising at least electrocardiogram (ECG) signals and a photoplethysmogram (PPG) signals. Step 404 comprises processing, by an artificial intelligence (AI) model 300 executed by the processor 202, the ECG signals and PPG signals. Step 406 comprises estimating, by the AI model, one or more demographic and anthropometric parameters based on processing the ECG and PPG signals. The method 300 may be executed by the processor 202 of the computing apparatus 200. The computing apparatus 200 may receive ECG and PPG signals from the EEG sensor 102 and PPG sensor 104.

FIG. 3 illustrates a further example of a computer-implemented method 500 for non-invasive cardiovascular health assessment. The method 500 commences at step 502. Step 502 comprises receiving ECG signals and PPG signals. Step 504 comprises preprocessing the received ECG signals and PPG signals. Signal preprocessing may comprise noise reduction, alignment ECG and PPG signals (i.e., waveforms) and normalization to ensure cross device consistency from sensor signals from multiple sensor types. Signal preprocessing can enhance quality of the received signals.

Preprocessing may include segmentation of raw ECG and/or PPG signals into fixed-length windows, resampling to achieve consistent frequency across sources, and normalization to mitigate variability between devices.

Step 506 comprises performing feature extraction of the pre-processed ECG signals and PPG signals. The feature extraction may be performed by the AI model 300. In one example, the AI model may apply machine learning (ML) and deep learning (DL) algorithms to identify waveform features correlated with demographic traits.

Step 508 comprises selecting one or more optimal features from the feature extraction. In one example, the AI model 300 may be programmed to select one or more optimal features. The AI model 300 may employ a Machine Learning approach or a Deep Learning approach to automatically extract meaningful features from the input signals.

Step 510 comprises applying an AI model e.g., machine learning on the optimal features. In one example, temporal learning may be applied by the AI model 300. In this example, the AI model 300 may use one or more recurrent neural networks (RNNs) or transformer architectures to capture dynamic, time-dependent patterns in the signals. Optionally ensemble methods may be executed by the AI model 300 to combine predictions from diverse model architectures, improving robustness and reliability. In one example, temporal processing may comprise capturing temporal dependencies and spatial relationships between the one or more optimal features.

Step 512 comprises estimating demographic parameters and anthropometric parameters based on the optimal features. The AI model 300 may be trained to estimate the demographic parameters and anthropometric parameters. The estimated demographic and anthropometric parameters may be presented on the user interface 212. The estimated parameters can provide a good indication of a subject's cardiovascular health.

The method 500 may also be used for additional clinical applications beyond demographic and anthropometric parameter estimation. The method may optionally comprise additional steps 514 and 516. Step 514 may comprise calculating, by the AI model, a cardiovascular age for the subject based on the estimated demographic and anthropometric parameters. Step 516 may comprise outputting the cardiovascular age and/or comparing the calculated cardiovascular age to the subject's chronological age to identify a risk of vascular aging. The method 500 may also comprise longitudinal tracking of health parameters to monitor aging progression or BMI fluctuations at step 518.

At step 516 the method may comprise comparing cardiovascular age vs. chronological age. Step 516 and/or step 518 can enable early identification of vascular aging and disease risk. The method 500 can also enable early detection of comorbidities, including hypertension, obesity, and predisposition to CVDs, based on demographic trends. Steps 514-518 may be optional steps.

In one example, the method 500 may comprise step 520. Step 520 may comprise tracking changes in estimated BMI or weight over time to detect abnormal trends and presenting estimated BMI or weight changes on a user interface.

The method 500 may optionally comprise generating a visual indicator (i.e., visual indicia) 522 to illustrate the estimate of the demographic and anthropomorphic parameters and presenting the visual indicator on a user interface 212. The visual indicator may comprise one or more of: a saliency map or heat map or attention heat map.

The computing apparatus 200 may optionally be configured to implement one or more Explainable AI (XAI) mechanisms or XAI models. The XAI models may be incorporated into the AI model 300. The XAI mechanisms may be provide the functionality to generate the visual indicators such as saliency maps or attention heatmaps. The visual indicators provide clinicians with interpretable insights into model decisions.

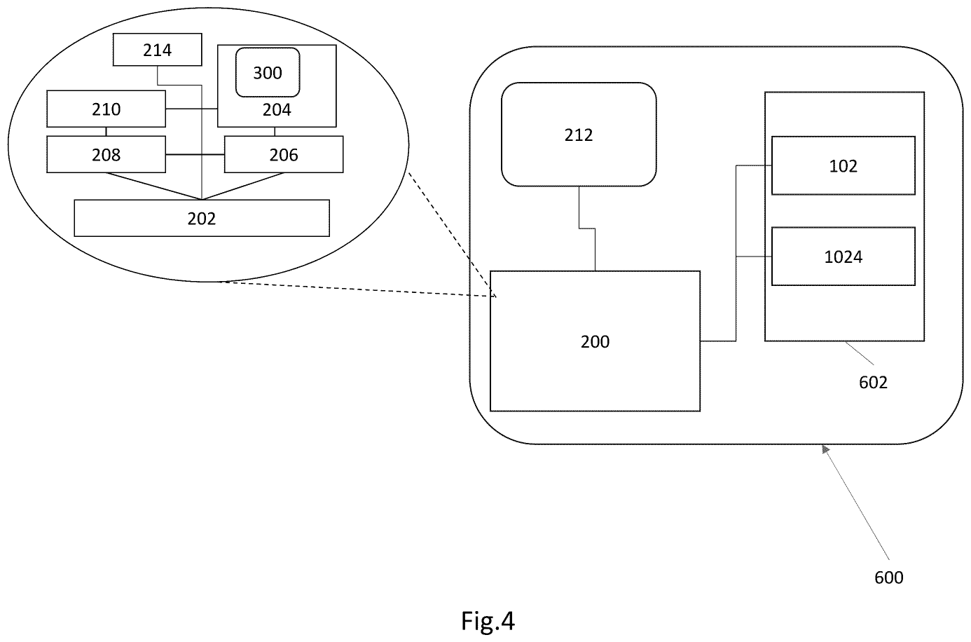

The system 100 and its components may be constructed as or implemented as a wearable device to allow for continuous patient (i.e., subject) monitoring, remote patient assessment and allowing for proactive care delivery. FIG. 4 illustrates a schematic diagram of an example wearable device 600 for use in non-invasive cardiovascular health assessment or cardiovascular monitoring. FIG. 4 illustrates an example embodiment of a wearable device 600. The wearable device 600 may comprise a sensor array 602 configured to continuously acquire ECG and PPG signals from a user. The sensor array 602 may comprise at least two types of sensors 102, 104. The sensors 102, 104 may be adapted to sense ECG signals and PPG signals respectively. The ECG sensor 102 may be a chest band or electrodes incorporated into the sensor array 602. The sensor array 602 may comprise a frame or a mounting structure and the electrodes may be mounted on the frame. The frame can be aligned with appropriate positions on a subject's body (i.e., patient's body) such that the ECG sensor 102 is in an operative position. The PPG sensor 104 may be a pulse oximeter or other PPG sensor. The PPG sensor 104 may be a fingertip mounted pulse oximeter or a wrist worn pulse oximeter.

The wearable device 600 comprises a computing apparatus 604. The computing apparatus 604 may comprise all the same components as computing apparatus 200 described earlier. The user interface i.e., a display may be a remote display for the wearable device. The computing apparatus 602 comprises a processor 606 and a memory unit 608, the processor 606 and memory unit 608 being operatively coupled to each other. The computing apparatus 604 may be operatively coupled to the sensor array 602 such that measured sensor signals can be received at the computing apparatus. The memory unit may be adapted to store an artificial intelligence (AI) model. The AI model used in the wearable device 600 may be AI model 300 as described herein. The AI model 300 is executable by the processor 606, the memory unit 608 further comprising instructions which, when executed by the processor 606 cause the processor to: receive physiological signals from a subject, wherein the physiological signals comprising at least electrocardiogram (ECG) signals and a photoplethysmogram (PPG) signals, process, by the AI model, the ECG signals and PPG signals, and; estimate, by the AI model, one or more demographic and anthropometric parameters based on processing the ECG and PPG signals. The demographic and anthropometric parameters may be displayed on a user interface.

In one example implementation the wearable device 600 may execute the method for non-invasive cardiovascular health monitoring 400 or 500 as described herein.

The AI model 300 as used in the system 100 and wearable device 600 will be described in more detail. In one example, the AI model comprising a recurrent neural network (RNN model), wherein the RNN model is trained to perform the steps of processing the received ECG and PPG signals and output an estimate of one or more demographic and anthropometric parameters.

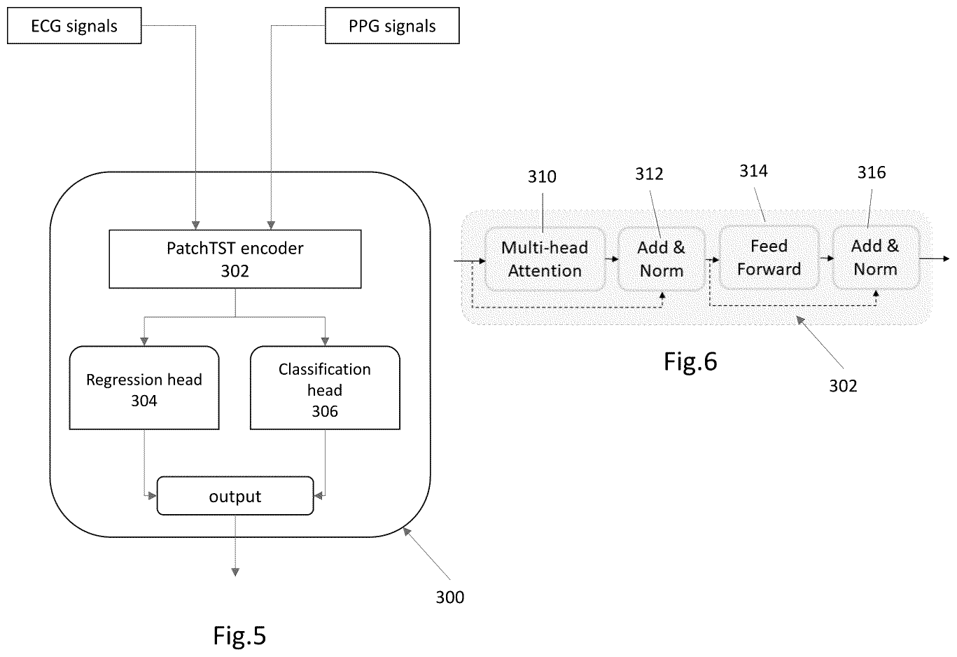

FIG. 5 illustrates an example architecture of the AI model 300. The AI model may comprise an encoder 302 and one or more task specific classification heads 304, 306. The encoder 302 may be trained to extract features from the ECG and/or the PPG signals. The task specific classification heads 304, 306 may be trained to apply regression to estimate an anthropometric parameter and/or classification to estimate a demographic parameter. The encoder 302 is coupled to the one or more task specific classification heads 304, 306 such that the output of the encoder is the input to each of the one or more classification heads, and the encoder is upstream of each of the one or more classification heads. In one example, the encoder may be pretrained. In one example, the encoder may be a PatchTST encoder. The model 300 may comprise a regression head 304 and a classification head 306. Each head leads to an output block. The output block may output the estimate of demographic and anthropometric parameters.

In one example, the regression head 304 may be configured to estimate anthropometric parameters such as age, weight, BMI, height etc. The classification head 306 may be configured to estimate demographic parameters such as gender.

FIG. 6 illustrates an example of architecture of the PatchTST encoder 302. The encoder 302 may comprise a multi head attention block 310 that is coupled to an Add & Norm block 312. The output of the multi head attention block 310 is fed into the Add & Norm block 312. Additionally, the inputs may also be fed forward into block 312. The Add & Norm block 312 may add the inputs with the outputs of the multi head attention and normalize the added data. The encoder 302 may further comprise a Feedforward block 314 operatively coupled to the Add & Norm block 312. The encoder may comprise a second Add & Norm block 316 that is adapted to add the outputs from the Feedforward block 314 and the input to the Feedforward block. The output of the encoder 302 may be extracted features from the ECG and PPG signals. Optionally the encoder 302 may also perform the preprocessing steps described herein.

In one example, the AI model may optionally comprise an ensemble architecture that combines multiple predictions from diverse model architectures, and; wherein the multiple model architectures may define the AI model. This can improve robustness and reliability. Ensemble learning may be incorporated to further improve robustness, and optional extensions such as self-supervised pretraining, explainable AI mechanisms, or integration of additional modalities (e.g., respiration or SpO₂) may be utilized to enhance performance.

The AI model 300 may be pretrained by employing a self-supervised learning method. The pretraining provides generalized feature representations that can be transferred to multiple downstream tasks without requiring full retraining. The AI model 300 may be pretrained on a large training dataset.

The training dataset comprises publicly available cardiovascular health assessment datasets that contain synchronized ECG and PPG signals, along with corresponding demographic and anthropometric labels such as age, gender, height, weight, and BMI. These datasets provide the necessary ground truth for learning the correlations between biophysical signals and individual characteristics through AI-driven analysis.

To ensure broad applicability and robust model training, the datasets employed encompass a diverse population, covering multiple age groups, genders, and a wide range of anthropometric values (BMI, weight, and height). This diversity helps in supporting generalization across varied patient cohorts and reducing potential biases in downstream predictions.

Optionally, the dataset may also be expanded with additional wearable sensor modalities such as respiration waveforms, oxygen saturation (SpO₂), or accelerometer data. These optional inputs can further improve the robustness of the system, especially in mobile or ambulatory monitoring scenarios.

As part of training the AI model 300 preliminary results were obtained using the PPG-BP dataset, a publicly available database of PPG signals accompanied by demographic and biometric information. This dataset was also used to evaluate the feasibility of predicting demographic and anthropometric parameters directly from PPG signals.

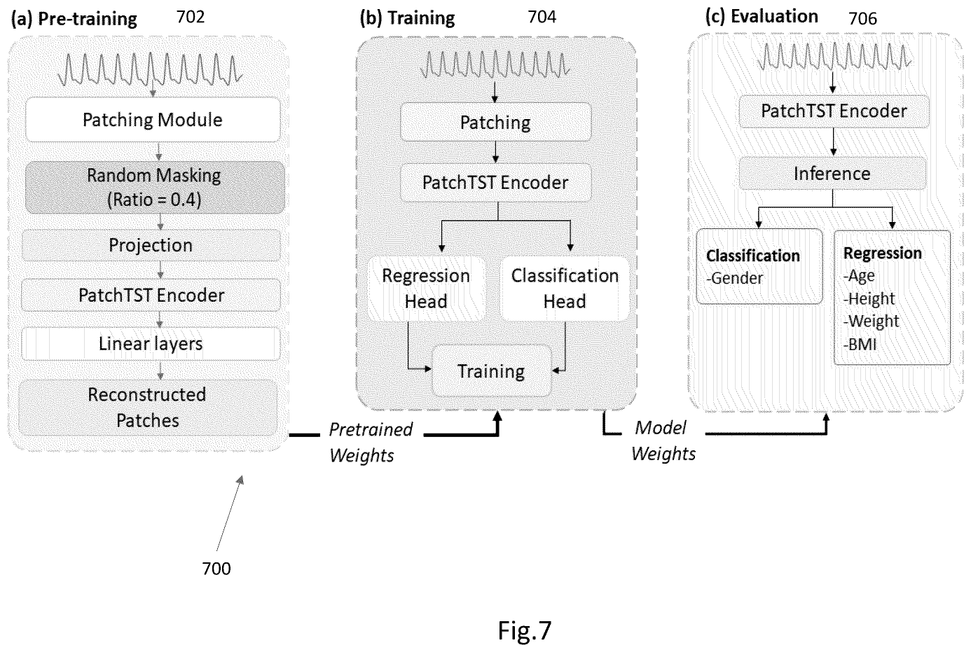

The feasibility of the AI model 300 was evaluated using the PPG-BP dataset, a publicly available database of PPG signals accompanied by demographic and biometric information. FIG. 7 illustrates a training and validation process of the AI model 300.

Referring to FIG. 7, the training and validation process 700 comprises a pretraining phase 702. During pretraining the system employs masked self-supervised learning (SSL) on the training data set. This enables robust feature learning from large-scale unlabeled data. A PatchTST-based architecture is used, where input time series segments are divided into patches and a subset of patches is randomly masked. The model 300 is trained to reconstruct the masked portions, thereby forcing the encoder 302 to capture temporal dynamics, waveform morphology, and latent physiological features. This pretraining strategy provides generalized feature representations that can be transferred to multiple downstream tasks without requiring full retraining. The pretraining phase is used to train the encoder 302. Pre-trained weights may be generated e.g., weights for the encoder.

During the pretraining phase 702, a patching module may be used to preprocess the labelled data. The masked portions may have a masking ratio of 0.4 for example. A projection module may segment the time series into patches. The linear layers may be used to reconstruct the patches.

Step 704 comprises training the AI model 300 with the training dataset. The training phase can be used to train the classification heads 304, 306 and the rest of the model 300. During the training phase the model weights are determined to enable the AI model 300 to estimate demographic and anthropometric parameters. The model weights may be stored and applied to the model.

Step 706 comprises evaluation i.e., validation of the AI model 300. After pretraining a, the model 300 is evaluated directly on labelled downstream tasks using demographic and anthropometric parameters as target outputs. In this phase, the pretrained encoder 302 extracts features from input PPG signals, and task-specific prediction heads 304, 306 are applied for regression (age, height, weight, BMI) or classification (gender).

Performance is assessed using standard regression and classification metrics. Mean Absolute Error (MAE) and Root Mean Square Error (RMSE) were used to assess the performance of regression tasks. F1-Score and Area Under the Receiver Operating Characteristic Curve (AUROC) may be used to assess performance of the classification tasks. Cross validation and statistical analysis ensure robustness of reported results yielding a more robust AI model 300.

As part of validating the AI model's feasibility, preliminary experiments were conducted by the inventors on the PPG-BP dataset. The pretrained AI model 300 including the PatchTST encoder was applied to PPG signals for demographic parameter estimation. The results are shown in Table 1 below.

TABLE I
Preliminary results of the proposed model on the PPG-BP

| | | |
|---|---|---|
| Tasks | MAE | RMSE |

Regression

| | | |
|---|---|---|
| Age | 13.28 | 15.95 |
| Height | 6.98 | 8.88 |
| Weight | 11.07 | 14.00 |
| BMI | 3.89 | 5.25 |

Classification

| Task | F1-Score | AUROC |
|---|---|---|
| Gender | 0.61 | 0.68 |

These results demonstrate the potential of the model 300 to accurately estimate demographic and anthropometric parameters using PPG signals alone. The findings of the testing provide strong preliminary evidence supporting the applicability and accuracy of the AI model-based approach described. The results also demonstrate the robustness of the AI model in estimating demographic and anthropometric parameters.

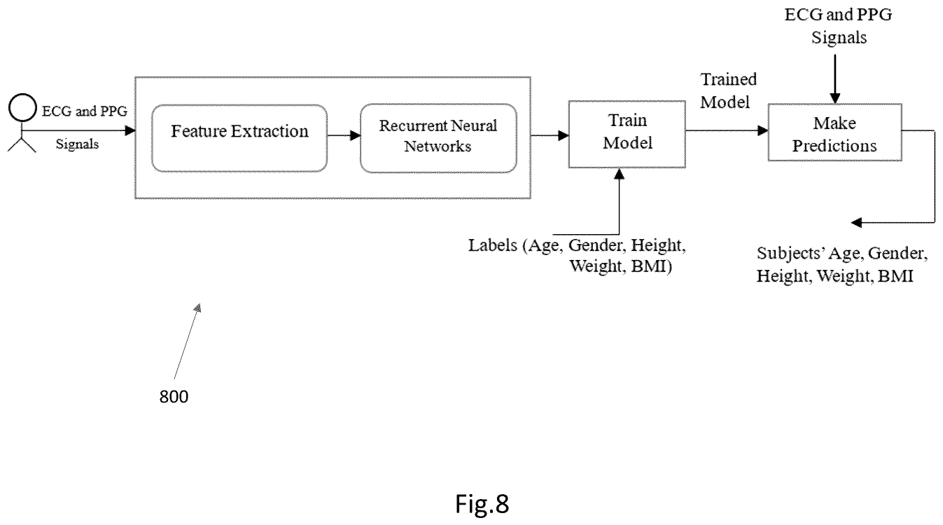

FIG. 8 illustrates a further alternative embodiment of a method of non-invasive cardiovascular health assessment. The method 800 may be executed by the system or wearable. The method 800 provides an AI based method for automatic feature extraction followed by the learning of features and making predictions based on learned features will be employed. The method 800 comprises collection and processing ECG and PPG signals, optimizing data quality through sophisticated preprocessing techniques. Machine learning models, including neural networks, then analyze the signals to estimate age, gender, height, weight, and BMI with unparalleled accuracy.

The method 800 utilized a publicly available CVD database, which contains ECG and PPG signals and demographic data. The data may be partitioned into training, validation, and test sets, and deep learning may be applied to carry out the automatic extraction of the highly significant features from the input signals, or machine learning may be applied for specific feature extraction. Subsequently, AI algorithms, such as recurrent neural networks, may train and optimize each model to estimate age, gender, height, weight, and BMI. The overall model may consist of a hybrid approach, using ML/DL (machine learning/deep learning) techniques for feature extraction, as well as recurrent neural networks to facilitate the temporal learning of time series waveforms. The implementation of rigorous assessment and cross-validation techniques provides for a robust and reliable performance. Following this, the trained model may undergo validation using ground truth data, addressing ethical considerations, and real-world integration possibilities, such as telemedicine applications and clinical trials, will be explored. Method 800 is an optional alternative.

The system, method and wearable as described can significantly transform the healthcare sector through its ability to facilitate remote monitoring, streamline clinical diagnoses, and augment preventative healthcare strategies. Furthermore, the smooth integration of this technology into telemedicine and wearable health equipment establishes it as a holistic solution within the modern healthcare environment.

By enabling accurate, real-time, and non-invasive prediction of these features, the system supports early risk identification, proactive interventions, and remote monitoring, while also offering broader applications in telemedicine, personalized healthcare, and even health insurance risk assessment.

The system 100 and method 400, 500 as described herein addresses this gap by introducing a non-invasive, AI-enabled system that integrates ECG and PPG signals to automatically estimate demographic features. The system 100 as described herein are advantageous because the system uses a non-invasive method of gathering ECG and PPG signals making gathering ECG and PPG signals easy and comfortable for subjects. The system 100 additionally uses a trained AI model to estimate the demographic and anthropometric parameters. These demographic and anthropometric parameters can be used to accurately determine cardiovascular health.

The system and method are advantageous because it applies a trained AI model that provides a transformative approach to estimating age, gender, height, weight, and BMI using ECG and PPG signals. The system and method use AI to provide improved accuracy in estimating age, gender, height, weight, and BMI from non-invasive ECG and PPG signals. This represents an advancement in the field of cardiovascular health engineering as it eliminates the need for invasive procedures or less precise methods. Unlike conventional approaches, this AI-based innovation provides a comprehensive profile of an individual's cardiovascular health in a single assessment. This holistic approach expedites diagnosis and provides a comprehensive assessment of the patient's cardiovascular health.

The system and method are also advantageous because they facilitate early detection of cardiovascular issues, which can empower healthcare practitioners to adopt proactive strategies that may prevent chronic cardiac disease progression and improve patient health. The continuous monitoring capabilities enabled by artificial intelligence (AI) offers real-time insights into the cardiovascular health state of patients, facilitating prompt interventions and modifications to treatment programs. The system can effectively be integrated with existing healthcare systems and electronic health records, facilitating prompt access to vital patient data for healthcare professionals. Consequently, this integration enhances the overall quality of treatment provided.

The system and method as described herein that uses an AI model for non-invasive cardiovascular health assessment has advantages that include real-time operation, clinical interpretability, scalability across devices and populations, and applicability to proactive healthcare management

Although not required, the embodiments described with reference to the Figures can be implemented as an application programming interface (API) or as a series of libraries for use by a developer or can be included within another software application, such as a terminal or personal computer operating system or a portable computing device operating system. Generally, as program modules include routines, programs, objects, components and data files assisting in the performance of particular functions, the skilled person will understand that the functionality of the software application may be distributed across a number of routines, objects or components to achieve the same functionality desired herein.

It will also be appreciated that where the methods and systems of the present invention are either wholly implemented by computing system or partly implemented by computing systems then any appropriate computing system architecture may be utilized. This will include stand alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Also, it is noted that the embodiments may be described as a process that is depicted as a flowchart, a flow diagram, a structure diagram, or a block diagram. Although a flowchart may describe the operations as a sequential process, many of the operations can be performed in parallel or concurrently. In addition, the order of the operations may be rearranged. A process is terminated when its operations are completed. A process may correspond to a method, a function, a procedure, a subroutine, a subprogram, etc., in a computer program. When a process corresponds to a function, its termination corresponds to a return of the function to the calling function or a main function.

Aspects of the systems and methods described above may be operable on any type of general-purpose computer system or computing device, including, but not limited to, a desktop, laptop, notebook, tablet, smart television, gaming console, or mobile device. The term “mobile device” includes, but is not limited to, a wireless device, a mobile phone, a smart phone, a mobile communication device, a user communication device, personal digital assistant, mobile hand-held computer, a laptop computer, wearable electronic devices such as smart watches and head-mounted devices, an electronic book reader and reading devices capable of reading electronic contents and/or other types of mobile devices typically carried by individuals and/or having some form of communication capabilities (e.g., wireless, infrared, short-range radio, cellular etc.).

In its various aspects, embodiments of the invention can be embodied in a computer-implemented process, a machine (such as an electronic device, or a general-purpose computer or other device that provides a platform on which computer programs can be executed), processes performed by these machines, or an article of manufacture.

1. A computer-implemented method for non-invasive Cardiovascular health assessment comprising:
receiving, at a processor, physiological signals from a subject,
wherein the physiological signals comprising at least electrocardiogram (ECG) signals and a photoplethysmogram (PPG) signals,
processing, by an artificial intelligence (AI) model executed by the processor, the ECG signals and PPG signals, and;
estimating, by the AI model, one or more demographic and anthropometric parameters based on processing the ECG and PPG signals.

2. The method of claim 1, wherein the step of processing comprising:
pre-processing the received ECG signals and PPG signals,
performing feature extraction of the pre-processed ECG signals and PPG signals,
selecting one or more optimal features, and;
wherein the demographic and anthropometric parameters are estimated based on the one or more optimal features.

3. The method of claim 2 wherein the step of processing comprising the step of performing temporal processing of the pre-processed ECG signals and PPG signals.

4. The method of claim 3, wherein temporal processing comprising capturing temporal dependencies and spatial relationships between the one or more optimal features.

5. The method of claim 1 wherein the demographic parameters comprising age, gender and the one or more anthropometric features comprising weight, height and BMI.

6. The method of claim 1 comprising
calculating, by the AI model, a cardiovascular age for the subject based on the estimated demographic and anthropometric parameters; and
outputting the cardiovascular age and/or comparing the calculated cardiovascular age to the subject's chronological age to identify a risk of vascular aging.

7. The method of claim 6, further comprising tracking changes in estimated BMI or weight over time to detect abnormal trends and presenting estimated BMI or weight changes on a user interface.

8. The method of claim 1, further comprising:
generating a visual indicator to illustrate the estimate of the demographic and anthropomorphic parameters,
presenting the visual indicator on a user interface, and;
wherein the visual indicator comprising one or more of: a saliency map or heat map or attention heat map.

9. The method of claim 3 wherein the AI model comprising a recurrent neural network (RNN model), wherein the RNN model is trained to perform the steps of processing the received ECG and PPG signals and output an estimate of one or more demographic and anthropometric parameters.

10. A system for non-invasive cardiovascular health monitoring comprising:
a computing apparatus comprising a processor and a memory unit, the processor and memory unit being operatively coupled to each other,
a user interface operatively coupled to or integrated into the computing apparatus,
the memory unit adapted to store an artificial intelligence (AI) model, wherein the AI model is executable by the processor, the memory unit further comprising instructions which, when executed by the processor cause the processor to:
receive physiological signals from a subject,
wherein the physiological signals comprising at least electrocardiogram (ECG) signals and a photoplethysmogram (PPG) signals,
process, by the AI model, the ECG signals and PPG signals, and;
estimate, by the AI model, one or more demographic and anthropometric parameters based on processing the ECG and PPG signals, and;
display the estimated demographic and anthropometric parameters on the user interface.

11. The system of claim 10, wherein the processor is programmed to:
pre-process the received ECG signals and PPG signals,
perform feature extraction of the pre-processed ECG signals and PPG signals,
select one or more optimal features, and;
wherein the demographic and anthropometric parameters are estimated based on the one or more optimal features.

12. The system of claim 11, wherein the processor is programmed to perform temporal processing of the pre-processed ECG signals and PPG signals.

13. The system of claim 12, wherein during temporal processing the processor is programmed to capture temporal dependencies and spatial relationships between the one or more optimal features.

14. The system of claim 10, wherein the demographic parameters comprise age, gender and the one or more anthropometric features comprising weight, height and BMI.

15. The system of claim 10, wherein the processor is programmed to:
calculate, by applying the AI model, a cardiovascular age for the subject based on the estimated demographic and anthropometric parameters; and
output the cardiovascular age and/or comparing the calculated cardiovascular age to the subject's chronological age to identify a risk of vascular aging.

16. The system of claim 15, wherein the processor is programmed to track changes in estimated BMI or weight over time to detect abnormal trends and present estimated BMI or weight changes on the user interface.

17. The system of claim 10, wherein the processor is further programmed to:
generate a visual indicator to illustrate the estimate of the demographic and anthropomorphic parameters,
present the visual indicator on the user interface, and;
wherein the visual indicator comprising one or more of: a saliency map or heat map or attention heat map.

18. The system of claim 11, wherein the AI model comprising a recurrent neural network (RNN model), wherein the RNN model is trained to perform the steps of processing the received ECG and PPG signals and output an estimate of one or more demographic and anthropometric parameters.

19. The system of claim 18, wherein the AI model is pretrained using a self-supervised learning framework on a dataset of unlabeled ECG signals, PPG signals, or both and, wherein the self-supervised learning framework is a masked self-supervised learning framework wherein segments of the unlabeled signals are masked and the AI model is trained to reconstruct the masked segments.

20. A wearable device for non-invasive cardiovascular health monitoring, comprising:
a sensor array configured to continuously acquire ECG and PPG signals from a user;
a computing apparatus, the computing apparatus comprising a processor and a memory unit, the processor and memory unit being operatively coupled to each other,
the computing apparatus being operatively coupled to the sensor array,
the memory unit adapted to store an artificial intelligence (AI) model, wherein the AI model is executable by the processor, the memory unit further comprising instructions which, when executed by the processor cause the processor to:
receive physiological signals from a subject,
wherein the physiological signals comprising at least electrocardiogram (ECG) signals and a photoplethysmogram (PPG) signals,
process, by the AI model, the ECG signals and PPG signals, and;
estimate, by the AI model, one or more demographic and anthropometric parameters based on processing the ECG and PPG signals.
